(19) United States
(12) Patent Application Publication
(10) Pub. No.: US 2008/0027271 A1
(43) Pub. Date: Jan. 31, 2008

(54) Methods for pelvic reconstruction and related implants

(76) Inventor: Joseph L. Maccarone, Voorhees, NJ (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/810,490

(22) Filed: Jun. 6, 2007

Related U.S. Application Data
(60) Provisional application No. 60/804,145, filed on Jun. 7, 2006.

(51) Int. Cl. A61F 2/02

(52) U.S. Cl. 600/30

(57) ABSTRACT

The present invention provides methods for vaginal reconstruction and implants used for such reconstruction. Generally, a first exemplary implant is preferably trapezoidal and anchored at six locations at the arcus tendineus in accordance with the present invention. A second exemplary implant preferably comprises a half circle shape and is anchored at two locations at the sacrospinous ligament in accordance with the present invention. The second implant may also optionally by anchored one or more times at the levator connective tissue.

### CROSS-REFERENCE TO RELATED APPLICATION

The present application claims priority under 35 U.S.C. §119(e) to U.S. Provisional Application No. 60/804,145, filed Jun. 7, 2006, which is incorporated herein by reference in its entirety.

### TECHNICAL FIELD

The present invention relates to pelvic reconstruction and implants used for such reconstruction.

### BACKGROUND

Medical conditions of urinary incontinence and pelvic prolapse are conditions of great importance. An aging population can be prone to such conditions.

Urinary incontinence and pelvic prolapse are related to tissues of the pelvic region such as the bladder, urethra, and the vaginal vault. Pelvic prolapse develops when intra-abdominal pressure, muscle failure, a surgical procedure such as a hysterectomy, or other factors, allow or cause a pelvic organ such as the vagina to become displaced. Within the general category of pelvic organ prolapse, specific types include vault prolapse (apical) such as enterocele; cystocele (anterior); rectocele (posterior); and combinations of these.

Various techniques have been designed to correct or ameliorate vaginal vault prolapse and its symptoms, with varying degrees of success. Nonsurgical treatments involve measures to improve the factors associated with prolapse, including treating chronic cough, obesity, and constipation. Other nonsurgical treatments may include pelvic muscle exercises or supplementation with estrogen.

A variety of surgical procedures have also been attempted for the treatment of pelvic conditions such as vaginal vault prolapse and urinary incontinence. See for example U.S. patent application Ser. No. 10/834,943, entitled “Method and Apparatus for Treating Pelvic Organ Prolapse,” filed Apr. 30, 2004, and Ser. No. 10/306,179, entitled “Transobturator Surgical Articles and Methods,” filed Nov. 27, 2002, the entireties of each of these two patent applications being incorporated herein by reference. Such patent applications describe articles and methods for treating incontinence and pelvic organ prolapse by use of a support member for supporting specific tissue. Application Ser. No. 10/834,943, for example, discusses a support member that includes a central tissue support portion and two extension portions, and related methods for implantation. The central tissue support portion can be attached at tissue of a prolapsed organ, e.g., at the posterior of the vaginal vault. The extension portions of the support member are then positioned through respective tissue pathways to place the support member in a therapeutic position for treatment of the prolapsed organ.

### SUMMARY

The present invention provides methods for pelvic reconstruction and implants used for such reconstruction. A first exemplary implant in accordance with the present invention is preferably trapezoidal and anchored at six locations at the arcus tendineus or obturator internus fascia. A second exemplary implant preferably comprises a half circle shape and is anchored at two locations at the ileococcygeous muscle or sacrospinous ligament in accordance with the present invention. The second implant may also optionally by anchored one or more times at the levator connective tissue.

Accordingly, in one aspect of the present invention, a method of single incision pelvic reconstruction is provided. The method comprises the steps of providing an implant comprising a support portion comprising biocompatible mesh material; creating a vaginal incision; inserting the implant through the vaginal incision and positioning the implant relative to tissue to be supported; attaching three attachment locations spaced apart along a first edge of the support portion of the implant at one or more of the arcus tendineus and the Obturator internus fascia; and attaching three attachment locations spaced apart along a second edge of the support portion of the implant at one or more of the arcus tendineus and the Obturator internus fascia.

In another aspect of the present invention, a method of single incision pelvic reconstruction is provided. The method comprises the steps of: providing an implant comprising a support portion comprising biocompatible mesh material, the support portion comprising a linear edge and an arcuate edge; creating a vaginal incision; inserting the implant through the vaginal incision and positioning the implant relative to tissue to be supported; and attaching two attachment location of the support portion of the implant at one or more of the ileococcygeous muscle and the sacrospinous ligament.

In another aspect of the present invention a pelvic implant is provided. The pelvic implant comprises a trapezoidal support portion comprising biocompatible mesh material, three extension portions spaced apart along a first edge of the support portion of the implant, each having a soft tissue anchor at a distal end thereof and capable of attaching the support portion at three spaced apart locations at the arcus tendineus, and three extension portions spaced apart along a second edge of the support portion of the implant, each having a soft tissue anchor at a distal end thereof and capable of attaching the support portion at three spaced apart locations at one or more of the arcus tendineus and Obturator internus fascia.

In yet another aspect of the present invention, a pelvic implant is provided. The pelvic implant comprises a support portion having a linear edge and an arcuate edge and comprising biocompatible mesh material, and two extension portions, each having a soft tissue anchor at a distal end thereof and capable of attaching the support portion at one or more of the ileococcygeous muscle and the sacrospinous ligament.

### BRIEF DESCRIPTION OF THE DRAWINGS

FIG. 1 is a schematic view an implant as implanted and anchored at six locations at the arcus tendineus in accordance with the present invention; and

FIG. 2 is a schematic view of another implant as implanted an anchored at two locations at the ileococcygeous muscle in accordance with the present invention.

### DETAILED DESCRIPTION

According the invention, a surgical implant is used to treat a medical condition, including the specific examples of implanting a support member (“implant”) to treat a pelvic condition such as vaginal vault prolapse or incontinence. Described herein are various features of surgical implants and surgical methods, useful for installing implants.

An implant can be implanted to treat disorders such as urge incontinence, mixed incontinence, overflow incontinence, functional incontinence, prolapse (e.g. vaginal), enteroceles (e.g. of the uterus), rectoceles, cystocele, and anatomic hypermobility.

In general, an implant can include a tissue support portion that can be used to support a pelvic tissue such as the urethra or vaginal tissue. During use, the tissue support portion is typically placed in contact with and attached to tissue to be supported, such as with a suture. An implant can additionally include one or more extension portions attached to the tissue support portion. See, e.g.: U.S. patent application Ser. Nos. 10/834,943; 10/306,179; U.S. Ser. No. 11/347,063, filed Feb. 3, 2006, entitled PELVIC IMPLANTS AND RELATED METHODS; U.S. Ser. No. 11/347,596, filed Feb. 3, 2006, entitled SURGICAL IMPLANTS AND RELATED METHODS AND SYSTEMS; and U.S. Ser. No. 11/346,750, filed Feb. 3, 2006, entitled TRANSOBTURATOR SURGICAL ARTICLES AND METHODS; the entireties of each of these being incorporated herein by reference.

An implant may include sections that are synthetic or of biological material (e.g., porcine, cadaveric, etc.). Extension portions may be, e.g., a synthetic mesh such as polypropylene. The tissue support portion may be synthetic (e.g., a polypropylene mesh) or biologic. Examples of support member products that are sold commercially include a number sold by American Medical Systems, Inc., of Minnetonka Minn., under the trade names Apogee® and Perigee® for use in treating pelvic prolapse (including vaginal vault prolapse, cystocele, enterocele, etc.), and Sparc®, Bioarc®, and Monarc® for treating urinary incontinence.

Exemplary implants can include a tissue support portion for placing in contact with tissue to be supported, and one or more “extension” portions, the tissue support portion being useful to support a specific type of pelvic tissue such as the urethra, bladder, or vaginal tissue (anterior, posterior, apical, etc.). The tissue support portion can be sized and shaped to contact the desired tissue when installed, e.g., as a sling, to contact and support tissue. A tissue support portion that is located between two or more extension or extension portions is sometimes referred to herein as a “central support portion.”

Extension portions are elongate pieces of material that extend from the tissue support portion and are connected to the tissue support portion, useful to attach to other anatomical features and thereby provide further support for the tissue support portion and the supported tissue. Multiple extension portions can extend from the tissue support portion as elongate “ends,” “arms,” or “extensions,” that are used to attach to other anatomy, such as by extending through a tissue path to an internal anchoring point.

Exemplary implants are preferably implanted by using tissue anchors to secure the implant in a desired position relative to tissue to be supported. A tissue anchor usable in accordance with the present invention preferably comprises a sharp portion that can pierce tissue and a holding portion such as a barb or the like that prevents the anchor from being pulled out of tissue after being implanted. The tissue anchor can be used to adjust the position of the implant as well as to secure the position of the implant. Permanent as well as dissolvable anchors can be used. See, e.g.: United States patent application publication number 2002/0161382, filed Mar. 25, 2002, entitled IMPLANT INSERTED WITHOUT BONE ANCHORS and United States patent application publication number 2004/0039453, filed Apr. 25, 2003, entitled PELVIC HEALTH IMPLANTS AND METHODS, the entireties of each of these being incorporated herein by reference.

Dimensions of an implant can be as desired and useful for any particular installation procedure or treatment, and to support a specific tissue or type of tissue. Exemplary dimensions can be sufficient to allow the tissue support portion to contact tissue to be supported, and to allow one or more extension portion to extend from the tissue support portion to a desired anatomical location to allow the extension portion be anchored to support the tissue support portion.

A tissue support portion should be sized and shaped to an overall area for contacting tissue being supported. The tissue support portion is of sufficient length to at least partially surround or otherwise be in contact with pelvic tissue to be supported. Various shapes and sizes are useful, and the particular shape can depend on the intended application, e.g., to treat incontinence, vaginal prolapse, etc. The shape of the tissue support portion can also be varied, depending on the intended application, and may be square, rounded, angled, rectangular, etc.

A width of an extension portion can be a width useful for implanting the implant and for providing desired strength and fixation properties during and after implantation and optional tensioning of the implant. Typical widths of extension portions can be in the range from 0.5 to 3 centimeters, e.g., from 0.8 to 2 centimeters. Extension portions can typically have a uniform or substantially uniform width along the length, normally not varying by more than about 25 percent of the average width along the length of the installed portion of the extension portion.

An example of a particular type of pelvic implant is the type that includes supportive portions including or consisting of a trapezoidal central support portion and six elongate extension portions extending from the central support portion. An example of another particular type of pelvic implant is the type that includes supportive portions including or consisting of a half-circle shaped central support portion and two elongate extension portions extending from the central support portion. The term “supportive portions” refers to portions of an implant that function to support tissue after the implant has been implanted, and specifically includes extension portions and tissue support portions.

According to particular embodiments, extension portions may be of the same or of different lengths. Extension portions of different lengths may be useful for certain types of surgical implantation procedures where one extension portion is inserted internally into a patient during an earlier step, and a second extension portion is inserted in a later step, optionally using the same tool for both steps.

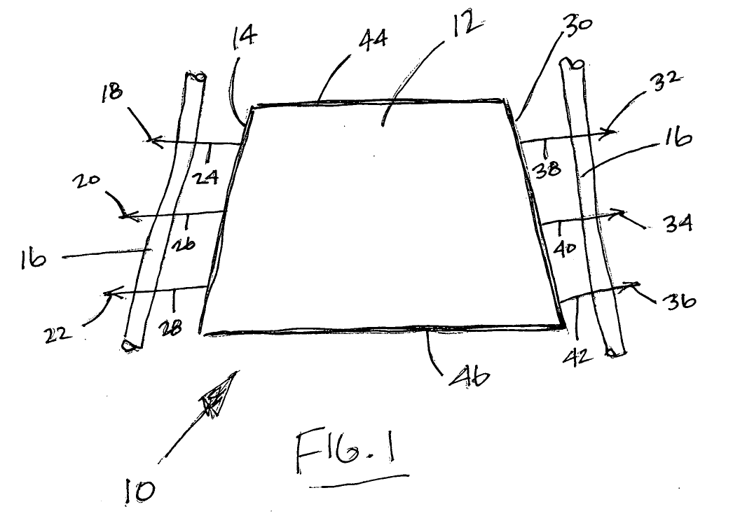

In FIG. 1 an implant 10 is schematically illustrated as implanted in accordance with the present invention. As shown, implant 10 includes support portion 12 comprising biocompatible mesh material which is positioned to support the desired tissue or organ (not shown). A first edge 14 of support portion 12 is attached at the arcus tendineus 16 (also referred to as the white line) by spaced apart anchors 18, 20, and 22 that are connected to first edge 14 by extension portions 24, 26, and 28 respectively. Similarly, a second edge 30 of support portion 12 is attached at the arcus tendineus 16 by spaced apart anchors 32, 34, and 36 that are connected to second edge 30 by extension portions 38, 40, and 42 respectively. Alternatively, first and second edges, 14 and 30, can be attached at the obturator internus fascia. Support portion 12 also comprises third edge 44 positioned at the anterior (pubis) and fourth edge 46 positioned at the posterior (ischial spine). Implant 10 can be provided in a kit which includes support portion 12, extension portions, anchors, 18, 20, 22, 32, 34, and 36, and at least one elongate insertion tool to surgically install the implant.

Preferably, as illustrated, support portion 12 is trapezoidal in shape. In a preferred embodiment the distance between the third edge 44 and fourth edge 46 is about 10 centimeters, e.g., from about 8 to 12 centimeters. Also, the length of the third edge 44 can be about 5 to 7 centimeters, and the length of the fourth edge 46 can be about 12 to 15 centimeters.

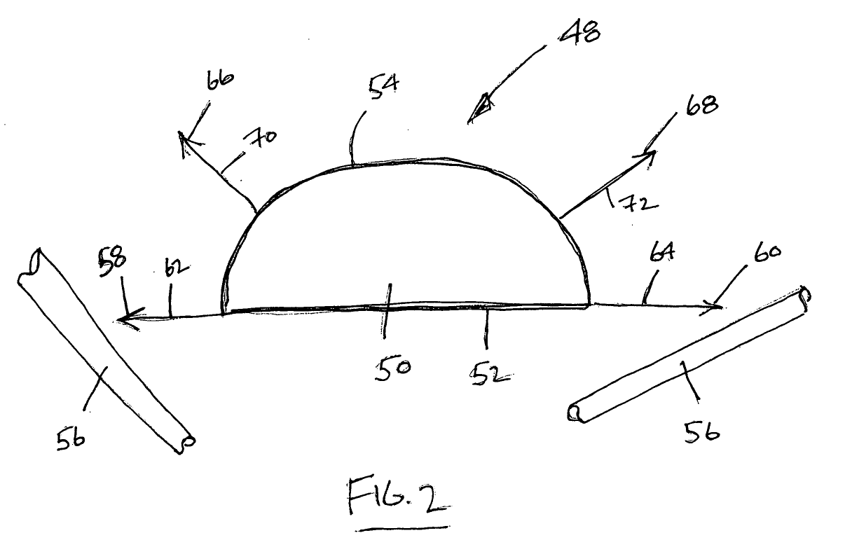

In FIG. 2 another implant 48 is schematically illustrated as implanted in accordance with the present invention. As shown, implant 48 includes support portion 50 comprising biocompatible mesh material positioned to support the desired tissue or organ (not shown). Support portion 50, as shown, comprises linear edge 52 and arcuate edge 54. Support portion 50 is attached at the ileococcygeous muscle near (e.g., within one centimeter from) sacrospinous ligament 56, by first and second anchors 58 and 60. Anchors 58 and 60 are connected to support portion 50 by extension portions 62 and 64, respectively. Optionally, as shown, arcuate edge 54 of support portion 50 can be attached at the levator connective tissue by anchors 66 and 68, which are connected to arcuate edge 54 by extension portions 70 and 72, respectively. As shown, edge 52 is positioned at the posterior and arcuate edge 54 is positioned at the anterior. Implant 48 can be provided in a kit that includes support portion 50, extension portions, anchors, and one or more insertion tools for surgical implantation of the implant.

Preferably, as illustrated, support portion 50 comprises a arcuate. The arcuate portion may be, e.g., a half-circle shape, an oblong oval or circle, or a combination of curved portions. In a preferred embodiment, arcuate edge 54 can be approximately a half-circle having a radius of about 7.5 centimeters, e.g., from about 6 to 9 centimeters. The length of edge 52 can be about 6 to 15 centimeters.

Implants 10 and 48 are preferably implanted transvaginally. In accordance with the present invention, a single incision is preferably used. In a typical procedure a vaginal incision is made, the implant is inserted through the vaginal incision and positioned relative to tissue to be supported. With respect to implant 10, the first edge 14 of support portion 12 is preferably attached at the arcus tendineus (and/or obturator fascia) with anchors 18, 20, and 22. The second edge 30 is then preferably attached at the arcus tendineus (and/or obturator fascia) on the opposite side with anchors 32, 34, and 36. Any suitable anchors and associated installation tools can be used. With respect to implant 48, support portion 50 is attached at the ileococcygeous muscle (near the sacrospinous ligament) with anchors 58 and 60. Optionally, the arcuate edge 54 can be attached at the levator connective tissue with anchors 66 and 68.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

What is claimed is:

1. A method of single incision vaginal reconstruction, the method comprising:
providing an implant comprising a support portion comprising biocompatible mesh material;
creating a vaginal incision;
inserting the implant through the vaginal incision and positioning the implant relative to tissue to be supported;
attaching three attachment locations spaced apart along a first edge of the support portion of the implant at the arcus tendineus; and
attaching three attachment locations spaced apart along a second edge of the support portion of the implant at the arcus tendineus.

2. The method of claim 1, wherein the support portion of the implant is trapezoidal.

3. The method of claim 2, wherein the first and second edges of the support portion are in the range from about 8 to about 10 centimeters long.

4. The method of claim 3, wherein an anterior edge of the support portion is about 5 to 7 centimeters long.

5. The method of claim 4, wherein a posterior edge of the support portion is about 12 to 15 centimeters long.

6. A method of single incision vaginal reconstruction, the method comprising:
providing an implant comprising a support portion comprising biocompatible mesh material, the support portion comprising a linear edge and an arcuate edge;
creating a vaginal incision;
inserting the implant through the vaginal incision and positioning the implant relative to tissue to be supported; and
attaching two attachment locations of the support portion of the implant at the ileococcygeous muscle.

7. The method of claim 6, wherein the support portion comprises a half-circle shape.

8. The method of claim 7, wherein the linear edge is about 12 to 15 centimeters long.

9. The method of claim 8, wherein the arcuate edge has a radius of about 8 to about 10 centimeters.

10. The method of claim 6, further comprising attaching at least one additional attachment location of the support portion at levator connective tissue.

11. A pelvic implant comprising:
a trapezoidal support portion comprising biocompatible mesh material;
three extension portions spaced apart along a first edge of the support portion of the implant, each having a soft tissue anchor at a distal end thereof and capable of attaching the support portion at three spaced apart locations at the arcus tendineus; and
three extension portions spaced apart along a second edge of the support portion of the implant, each having a soft tissue anchor at a distal end thereof and capable of attaching the support portion at three spaced apart locations at the arcus tendineus.

12. The implant of claim 11, wherein the first and second edges of the support portion are from about 8 to about 12 centimeters long.

13. The implant of claim 12, wherein an anterior edge of the support portion is from about 5 to about 7 centimeters long.

14. The implant of claim 13, wherein a posterior edge of the support portion is from about 12 to about 15 centimeters long.

15 A pelvic implant comprising:
a support portion having a linear edge and an arcuate edge and comprising biocompatible mesh material; and
two extension portions, each having a soft tissue anchor at a distal end thereof and capable of attaching the support portion at the ileococcygeous muscle.

16. The implant of claim 15, further comprising at least one additional extension portion having a soft tissue at a distal end thereof and capable of attaching the support portion at levator connective tissue.

17. The implant of claim 15, wherein the support portion comprises a half-circle shape.

18. The implant of claim 17, wherein the linear edge is from about 12 to about 15 centimeters long.

19. The implant of claim 18, wherein the arcuate edge has a radius of about 8 to about 12 centimeters.
